(19) United States
(12) Patent Application Publication
(10) Pub. No.: US 2026/0363591 A1
(43) Pub. Date: Nov. 19, 2026

(54) TEST METHOD FOR EVALUATING MIND/BODY CONDITION

(71) Applicants: Good House, Inc, Tokyo (JP); META CONSCIOUS CO., LTD., Tokyo (JP)

(72) Inventors: Atsushi ONO, Tokyo (JP); Koji SUGIURA, Tokyo (JP)

(73) Assignees: Good House, Inc, Tokyo (JP); META CONSCIOUS CO., LTD., Tokyo (JP)

(21) Appl. No.: 19/477,487

(22) PCT Filed: Apr. 27, 2023
(86) PCT No.: PCT/JP2023/016750

§ 371 Date: Oct. 22, 2025

(51) Int. Cl. A61B 5/16; A61B 5/00; A61B 5/11

(52) U.S. Cl. CPC A61B 5/165; A61B 5/1122; A61B 5/7405; A61B 5/7435

(57) ABSTRACT

In a test method for determining a mental and physical condition using an O-ring test, the test method uses a portable terminal and an application. The application measures a pressing force and a mobility of a thumb of a subject from data acquired by the portable terminal, and determines a mental and physical condition of the subject from a measurement result.

### TECHNICAL FIELD

The present invention relates to a method for testing a mental and physical condition of a subject. Specifically, the present invention relates to a test method that can determine the mental and physical condition of the subject from mobility of a finger of the subject.

### BACKGROUND ART

It is known that a motion function of a finger of a human reflects the mental and physical condition or a health condition of the human. In the related art, in a field of clinical medicine and care welfare, a health condition of a subject has been estimated from a grip strength or a writing ability of the subject.

An O-ring test is a method for determining the health condition of the subject based on the magnitude of a pressing force (so-called finger pressure) applied between a thumb and a forefinger, and is particularly used in the clinical practice of oriental medicine. The history of the O-ring test is long, and various proposals have been made regarding the O-ring. Note that the term “finger pressure” stated here is different from the power of an acupressure or the strength of a massage used in a massage scene.

For example, Non Patent Document 1 discloses practicality of the O-ring test and a method for improving the practicality. A method for accurately performing the O-ring test with a simple device has also been proposed. Patent Documents 2, 3, 4, 5, and 6 disclose an inspection instrument for performing the O-ring test by one subject. Patent Document 7 discloses a method of combining the O-ring test with a saliva inspection to convert a result of the O-ring test into a more objective numerical value and utilize the result.

As described in the citation lists, a dedicated inspection equipment has been required for the O-ring test and a developed test thereof. Although an individual inspection equipment is downsized and simplified, it is not convenient for use in a home or a workplace or for carrying.

Currently, it is possible for each of the general public with poor expertise to acquire his or her sensor data with high accuracy at desired time and place and to determine his or her health condition by using a portable terminal such as a smartphone or a smartwatch. Already, the subject can self-evaluate a blood pressure, a pulse rate, a body temperature, a blood oxygen concentration, or the like by using the smartphone or the like.

The result of the O-ring test reflects not only the physical health condition of the subject but also stress and self-affirmation. From the viewpoint that both a physical condition and a mental condition of the subject can be evaluated by the O-ring test, it is possible to estimate the life satisfaction and the comprehensive mental and physical quality of the subject unlike the evaluation using sensor data such as the blood pressure, the pulse rate, the body temperature, and the blood oxygen concentration.

In this case, it is ideal if the individual can perform the O-ring test at desired time and place without special test equipment.

### CITATION LIST

### Non Patent Document

Non Patent Document 1: “Problems with the O-Ring Test and the New Vader Quantum Test Technique” by Hidetoshi Tachibana, Tama Publishing, issued on Jun. 17, 2020, ISBN: 978-4812704387

### Patent Document

Patent Document 2: Japanese Registered Utility Model No. 3009292

Patent Document 3: Japanese Unexamined Patent Application Publication No. H7-289525

Patent Document 4: Japanese Unexamined Patent Application Publication No. H7-289541

Patent Document 5: Japanese Unexamined Patent Application Publication No. 2002-85388

Patent Document 6: Japanese Unexamined Patent Application Publication No. 2003-47603

Patent Document 7: Japanese Unexamined Patent Application Publication No. 2015-208666

### SUMMARY OF INVENTION

### Problem to be Solved by Invention

Therefore, the present inventor has searched for a test method in which the O-ring test can be performed by one subject without using special test equipment.

### Means for Solving Problem

As a result, the present inventor has developed a test method that can be performed by one subject by using a portable terminal such as a smartphone as a development type of the O-ring test, and an application used for the test.

According to the present invention, there is provided a method for determining a mental and physical condition of a subject based on a measurement value corresponding to a movable range and a pressing force of a thumb of the subject. In addition, the present invention is an application that measures the movable range and the pressing force of the thumb of the subject to determine the mental and physical condition of the subject. In the present invention, the term “mental and physical condition” is broadly defined to include, for example, a mental and physical condition that reflects a preference of food and hobbies, and a tendency and an intensity of a subconsciousness, a desire, and a motivation. That is, the present invention includes the following inventions.

### Invention 1

A test method using a portable terminal (1) and an application (2), the test method including the following steps.
Step 1: the application (2) starts an operation for a test.
Step 2: a subject (3) responds to a question and/or invitation message (21), and then slides a ball (32) of a thumb (31) of a dominant hand of the subject (3) on a surface of a touch panel (11). Here, the touch panel (11) has a display function and a sensor function.
Step 3: the application (2) measures a dimension (41) of a contact trajectory (4) between the ball (32) of the thumb (31) that appears in Step 2 and the touch panel (11).

### Invention 2

The test method according to Invention 1, in which in Step 1, the application (2) outputs the question and/or invitation message (21) for making a request for a response of the subject (3), and

the test method further includes the following Step 4 after Step 3.
Step 4: the application (2) compares the dimension (41) with a preset reference value (40) to generate answer data (5) of the subject (3) to the question and/or invitation message (21).

### Invention 3

The test method according to Invention 1, in which in Step 1, the application (2) outputs a question and/or invitation message (21) for making a request for a response of the subject (3), and
the test method further includes the following Step 4 and Step 5 after Step 3.
Step 4: the application (2) compares the dimension (41) with a preset reference value (40) to generate answer data (5) of the subject (3) to the question and/or invitation message (21).
Step 5: the application (2) determines a mental and physical condition (7) of the subject (3) based on performance data (6) calculated from one or more pieces of answer data (5) obtained by executing Steps 1 to 4 one or more times.

### Invention 4

The test method according to Invention 1, in which in Step 3, a length (411) of the contact trajectory (4) is measured, and
in Step 4, the application (2) compares the length (411) with a preset reference length (4110) to generate answer data (5) of the subject (3) to the question and/or invitation message (21).

### Invention 5

The test method according to Invention 4, in which the length (411) corresponds to a total length of the contact trajectory (4).

### Invention 6

The test method according to Invention 4, in which the length (411) corresponds to a shortest distance between both end points of the contact trajectory (4).

### Invention 7

The test method according to Invention 1, in which in Step 3, a turning angle (412) of the thumb (31) is measured from the contact trajectory (4), and
in Step 4, the application (2) compares the turning angle (412) with a preset reference turning angle (4140) to generate answer data (5) of the subject (3) to the question and/or invitation message (21).

### Invention 8

The test method according to Invention 1, in which in Step 2, the subject (3) responds to the question and/or invitation message (21), and then slides the ball (32) of the thumb (31) of the dominant hand of the subject (3) on the surface of the touch panel (11) to drum roll a scale displayed on the touch panel (11),
in Step 3, a scale movement (413) by the drum roll in Step 2 is measured as the dimension (41), and
in Step 4, the application (2) compares the scale movement (413) with a preset reference scale movement (4150) to generate answer data (5) of the subject (3) to the question and/or invitation message (21).

### Effects of Invention

The present invention is a method for diagnosing a mental and physical condition of a subject with simple operations, anytime, anywhere, and actively, sometimes even as entertainment. The test method and application of the present invention are not found in a related field of an O-ring test in the related art, and are innovative.

### BRIEF DESCRIPTION OF DRAWINGS

FIG. 1 An aspect in which a subject (3) holds a portable terminal (1) according to the present invention.

FIG. 2 An aspect in which a thumb (31) of the subject (3) in the present invention comes into contact with the portable terminal (1).

FIG. 3 An example of a contact trajectory (4) of the present invention.

FIG. 4 An example of a contact trajectory (4) of the present invention.

FIG. 5 A display example of a smartphone (101) used in the present invention.

FIG. 6 An aspect in which the subject (3) slides the thumb (31) in the present invention.

FIG. 7 An aspect in which the subject (3) slides the thumb (31) in the present invention.

FIG. 8 An aspect in which the subject (3) slides the thumb (31) in the present invention.

FIG. 9 An aspect in which the subject (3) slides the thumb (31) in the present invention.

FIG. 10 An aspect in which the subject (3) slides the thumb (31) in the present invention.

FIG. 11 An aspect in which the subject (3) slides the thumb (31) in the present invention.

### EMBODIMENTS FOR IMPLEMENTING INVENTION

### Test Method (100)

A test method (100) of the present invention is executed by a portable terminal (1) and an application (2). Steps that are always executed in the test method (100) are Steps 1, 2, and 3 to be described later. The test method (100) preferably includes the following Steps 1, 2, and 3. The test method (100) preferably further includes the following Steps 1, 2, 3, and 4, or Steps 1, 2, 3, 4, and 5 to be described later.

The application (2) used in the present invention refers to a program or a program set for executing the test method (100) of the present invention, and an installation destination is not limited. Any of a so-called web application and a mobile application corresponds to the application (2). A pressing force and a mobility of a thumb (31) of a subject (3) are measured from data that is acquired by the portable terminal (1), and a mental and physical condition of the subject (3) is determined from the measurement result.

The portable terminal (1) includes a touch panel (11) having a display function and a sensor function. The portable terminal (1) used in the present invention is not particularly limited, but a smartphone or a tablet that is convenient for the subject (3) to carry is preferable. As the touch panel (11), a type of a display screen that is commonly used as a display unit of a smartphone or a tablet and that is operated by a user by bringing a finger or a pen-type input device into contact therewith can be used without limitation.

Specifically, the data acquired by the portable terminal (1) is a trajectory along which a contact point between the thumb (31) and the touch panel (11) of the portable terminal (1) moves during a period of a response operation of the subject (3).

The application (2) calculates a dimension of the contact trajectory (4) from the data of the contact trajectory (4). When a shape dimension is equal to or larger (longer) than a reference value, the application (2) determines that the pressing force and the mobility of the thumb (31) are high and that the mental and physical condition is good. When the shape dimension is smaller (shorter) than the reference value, the application (2) determines that the pressing force and the mobility of the thumb (31) are low and the mental and physical condition is not good.

The application (2) may operate in a state of being downloaded to the portable terminal (1), or may operate in a state of being stored in a medium such as a cloud on the Internet while communicating with the portable terminal (1) connected to the Internet.

When the test is performed in accordance with the test method of the present invention, in reality, the subject (3) holds the portable terminal (1) before Step 1. Here, the subject (3) holds the portable terminal (1) in a state in which the subject (3) can turn the thumb (31) around a base of the thumb (31) of a dominant hand and can bring a ball (32) of the thumb (31) into contact with a surface of the touch panel (11) during the turning.

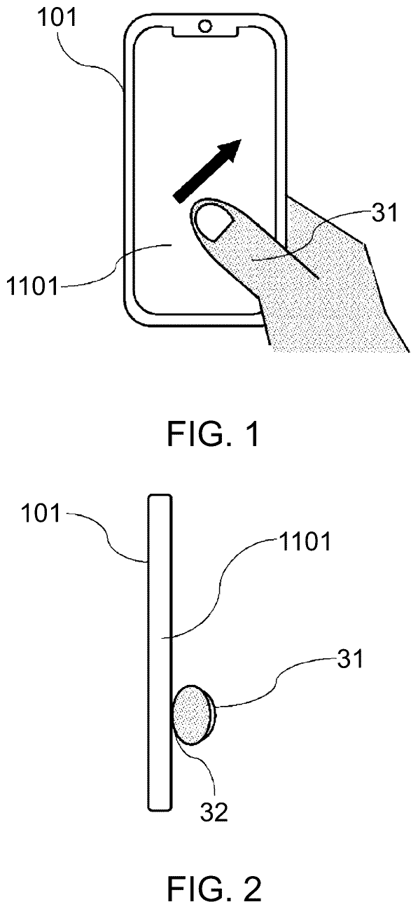

In short, as shown in FIGS. 1 and 2, the subject (3) waits for a test start in a state in which the ball (32) of the thumb (31) is in contact with the surface of the touch panel (11) and the thumb (31) can turn. FIGS. 1 and 2 show a case where the subject (3) is right-handed. FIGS. 1 and 2 are schematic views, and details are exaggerated or omitted. A part of the hand of the subject (3) is omitted. FIG. 1 shows a positional relationship between a display screen (1101) of a smartphone (101) and the thumb (31). An arrow indicates a direction in which the thumb (31) turns. FIG. 2 shows a positional relationship between the smartphone (101), the display screen (1101), and the ball (32) of the thumb (31).

### Step 1 of Test Method (100)

Step 1 is a step in which a start operation is performed in the test method (100). Typically, in Step 1, the application (2) outputs a question and/or invitation message (21) to the subject (3) on the touch panel (11) of the portable terminal (1). The above-described output is not limited as long as the output is in a form that can be recognized by the subject (3). For example, the message (21) is output as a character or an image to the touch panel (11) of the portable terminal (1) or as a voice toward the subject (3).

The application (2) can provide a practice stage in which the subject (3) responds to the message (21) before Step 1 or in parallel with Step 1. In such a practice stage, particularly, it is preferable that the message (21) is a question that allows the subject (3) to have a feeling of “affirmative (Yes) (I think so)” or “negative (No) (I do not think so)” and to clearly determine and distinguish the will. Preferably, the message (21) guides the subject (3) such that a relatively long contact trajectory (4) appears in Step 3 described later when the subject (3) has no problem in the mental and physical condition. Alternatively, the message (21) preferably guides the subject (3) such that the relatively long contact trajectory (4) appears in Step 3 described later in a case of a positive content for the mental and physical condition of the subject (3).

The message (21) may be a question that the subject (3) asks himself/herself or a message that encourages or consoles himself/herself. For example, the message may be a message that prompts the subject (3) to ask and answer himself/herself, such as “Today is a lucky day for you in the constellation of Aries! Let's cheer for yourself before going to work!” and “Do you still have the same feeling for your favorite character? Ask yourself first thing in the morning.”

For example, the application (2) can customize the question and/or invitation message (21) in correspondence with a condition input by the subject (3). For example, the application (2) displays a basic form of the message (21): “How much your pet wants to see you” on the portable terminal (1) (touch panel (11)), and the subject (3) can be provided with a customized message (21): “Your pet**'s ***-chan is waiting for you at home. Imagine how happy ***-chan would be when your pet greets you at the front door and slide your finger!” based on input information “Tama-chan of the cat” from the subject (3).

In the test method of the present invention, the message (21) may be a content in which the subject (3) answers his/her own question, a display on the touch panel (11), or a voice received by the subject (3) from the portable terminal (1).

In Step 1, for example, when “Do you put strength in your fingertip?” is output as the message (21), the subject (3) places the thumb (31) on a circular frame designated as the starting point on the touch panel (11). Next, “Slide your thumb from the start circular frame to a goal position with a minimum force.” is displayed as the message (21). In Step 2 described later, the subject (3) slides the ball (32) of the thumb (31) of the hand holding the touch panel (11) toward the goal circular frame from the start circular frame.

In addition, for example, the text or voice of “Slide your thumb immediately after asking a question while being conscious of ***-chan.” is displayed or output to the portable terminal (1) as the message (21).

A message that prompts a response operation of the subject (3) can be displayed on the touch panel (11) in Step 1. As such a message, for example, a text such as “Move the thumb in a direction of an arrow while imaging the answer to this question.” and “Finger answer start!” can be displayed on the touch panel (11). Instead of or in addition to such a message, a picture or a photograph can be displayed. Such a message can also be output as an audio message by using an audio function of the portable terminal (1).

In addition, the application (2) may output the question and/or invitation message (21) after detecting that the thumb (31) of the subject (3) has come into contact with the touch panel (11). In this case, the subject (3) receives the question and/or invitation message (21) in a state in which the ball (32) of the thumb (31) is pressed against the touch panel (11), and proceeds to Step 3 described below. That is, the subject (3) responds to the question and/or invitation message (21), and then starts a turning motion of the thumb (31) that has been stationary until then.

### Step 2 of Test Method (100)

In Step 2, the subject (3) responds to the question and/or invitation message (21), and then slides the ball (32) of the thumb (31) of the dominant hand of the subject (3) on the surface of the touch panel (11). Here, the touch panel (11) has a display function and a sensor function. The ball (32) of the thumb (31) starts to move while the ball (32) of the thumb (31) is in contact with the surface of the touch panel (11), moves by a certain distance, and then stops on the touch panel (11) or moves away from the surface of the touch panel (11). In the present invention, the entire contact points between the ball (32) of the thumb (31) sensed by the portable terminal (1) in Step 2 and the touch panel (11) are referred to as a contact trajectory (4).

In Step 2, since the thumb (31) of the hand of the subject (3) which holds the portable terminal (1) slides on the touch panel (11), a motion range of the thumb (31) is limited. The following types are mainly assumed as the motion of the ball (32) of the thumb (31) in Step 2.
Draw a fan-shaped arc centered on the vicinity of the base of the thumb (31) or the vicinity of the wrist. The ball (32) of the thumb (31) is stationary on the surface of the touch panel (11) and an ending point appears, or is separated from the surface of the touch panel (11) while continuing the turning motion.
The ball (32) of the thumb (31) moves while drawing a curve that is not a straight line and/or a fan shape. The ball (32) of the thumb (31) is stationary on the surface of the touch panel (11) and the ending point appears, or moves away from the surface of the touch panel (11) while moving.

As in the above-described example, when the terms “start” and “goal” of the thumb (32) are previously indicated to the subject (3), a change is made in accordance with positions of the terms “start” and “goal” displayed on the touch panel (11). For example, as shown in FIG. 6, when the start (A) and the goal (B) are displayed along a diagonal line of the display screen (1101) of the smartphone (101) facing the subject (3), the subject (3) turns the thumb (31) placed at the start (A) around the center of the base and slides the ball (32) of the thumb (31) to the goal (B). At this time, the contact trajectory (4) similar to a part of an arc is obtained.

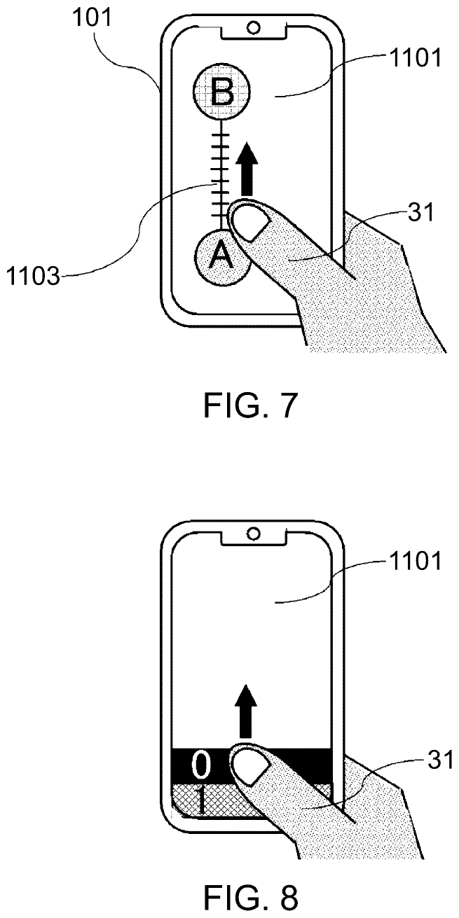

In addition, for example, as shown in FIG. 7, when the start (A) and the goal (B) are arranged on upper and lower sides of the display screen (1101), the subject (3) moves the thumb (31) to flip-up starting from the base. At this time, the contact trajectory (4) is generally a straight line extending up and down near the start (A). Generally, the straight line terminates away from the start (A) or reaches an ending point by changing a shape in a curved manner near the goal (B). Further, in order to guide the operation of the subject (3), the application (2) can display a scale (1103) connecting the start (A) and the goal (B) as shown in FIG. 7.

The touch panel (11) can perform various displays in addition to the scale to prompt the subject (3) to perform the slide motion of the thumb (31). For example, it is possible to display display areas in different colors in correspondence with a distance from the starting point, to display a plurality of target points in which scales 0, 1, 2, 3, and the like are displayed in correspondence with the extent of a movement distance, and the like.

According to findings related to the O-ring test, it is known that a pressing force of a finger tends to increase and/or a movable range of the finger tends to widen when a person is in a state in which a positive emotion is increased or when a level of physical or psychological stress is low. In Step 2, when the subject (3) slides the thumb (31) in a state of having a high degree of positive emotion to the question and/or invitation message (21), the degree of contact between the thumb (32) and the touch panel (11) increases. Therefore, the contact trajectory (4) is curved or linear, but the entire contact trajectory (4) is lengthened, or the shortest distance between both end points of the contact trajectory (4) is lengthened.

The subject (3) can retry the operation after the subject (3) once slides the ball (32) of the thumb (31) while being in contact with the surface of the touch panel (11).

For example, the application (2) can display a message that prompts selection with a text set of “Have you completed the slide?”, “Yes/Next”, and “No/Retry” on the touch panel (11). In this case, when “Yes/Next” is selected (the subject (3) inputs selection data to the portable terminal (1) by touching the screen), the application (2) terminates Step 2.

In this case, when “No/Retry” is selected (when the subject (3) inputs selection data to the portable terminal (1) by touching the screen), the application (2) waits for a new contact to be made in order to repeat the step, and when the new contact is sensed, “Slide your thumb from the circular frame of the start to the goal position with a minimum force.” is displayed again. In this way, the subject (3) can repeat Step 2.

In Step 2, the application (2) can output a message to the subject (3) in correspondence with a motion state of the thumb (31) during a period from the start to the end of the turning of the thumb (31). For example, it is possible to generate a vibration to distinguish a voice output according to the motion state of the thumb (31) to assist the operation of the blind subject (3).

In Step 2, a so-called drum roll method can be adopted when the ball (32) of the thumb (31) of the dominant hand of the subject (3) is slid on the surface of the touch panel (11).

The term “drum roll” is also referred to as a picker method, and is a method that is often used when selecting one from a large number of candidates displayed on a smartphone and inputting the selected one. For example, the method is used for selection and input of a date, a time, an age, a place name, and the like. As if a roll printed on a surface is rotated to show a desired printing, a user scrolls an initial display in two directions (for example, up and down) for change to a desired input item. In the smartphone, a drum roll application often generates a sound effect in accordance with a change in display and causes the user to image the rotation of the roll. A distance (magnitude of the rotation of the roll) from the initial display to a target display corresponds to a speed of the scroll.

When the drum roll method is used in Step 2, the application (2) prompts the subject (3) to advance (rotate) the scale initially displayed on the touch panel (11) in response to the question and/or invitation message (21). The displayed scale is displayed to advance in one direction (for example, an upward direction of the touch panel (11)). The subject (3) slides the ball (32) of the thumb (31) of the dominant hand on the surface of the touch panel (11) to drum roll the scale displayed on the touch panel (11).

In the present invention, a wide range of so-called drum roll methods from a basic method to a usual method in Step 2 can be used. When the drum roll method is adopted in Step 2 of the present invention, a change in the scale is larger as the ball (32) of the thumb (31) is forcefully slid.

### Step 3 of Test Method (100)

In Step 3, the application (2) measures a dimension (41) of the contact trajectory (4) between the ball (32) of the thumb (31) and the touch panel (11) which appears in Step 2.

The contact trajectory (4) is based on position information detected by a method effective for a touch panel. A detection method used here may be any of a surface type capacitance method, a low hard film method, and an infrared ray method in addition to a projection type electrostatic capacitance method that is widely adopted in smartphones, tablets, and the like. According to the above-described typical methods, the dimension (41) does not directly depend on a speed at which the ball (32) of the thumb (31) moves on the surface of the touch panel (11), and depends on a distribution of positions detected as “contact (touch)” in each method. Therefore, even when there is a time for the ball (32) of the thumb (31) to stop once while moving on the surface of the touch panel (11), the contact trajectory (4) is detected.

When the detected contact trajectory (4) is displayed on the touch panel (11) as a line that changes from time to time in Step 2, the detected contact trajectory (4) can be displayed as a trajectory in an X-direction and a Y-direction by using a swipe detection method of a smartphone.

When the dimension (41) deviates from a range set in the application (2) in advance as an allowable range, for example, when the above-described contact state is not detected within a certain time, when the dimension (41) exceeding the allowable range is detected due to a case where the subject (3) drops the portable terminal (1) and the thumb (31) is pressed to the outside of the frame of the touch panel (11), and the like, the application (2) can display a message prompting the subject (3) to slide the finger again on the touch panel (11). In this case, the test returns to Step 1 or Step 2 and continues.

Step 3 is terminated when the dimension (41) is within a range set in the application (2) as an allowable range in advance. In Step 3, a measurement result of the dimension (41) can also be registered in a database controlled by the application (2).

The measured dimension (41) is not limited as long as the measured dimension (41) is a characteristic value reflecting the pressing force and/or degree of the movable range of the thumb (31). The measured dimension (41) is actually a calculation value calculated by a calculation method in which an appropriate statistical method is introduced into two-dimensional coordinates of points belonging to the contact trajectory (4). For example, a length (411) of the contact trajectory (4) can be measured as the dimension (41).

The length (411) may be a total length of a line that is the contact trajectory (4).

In this case, the application (2) calculates the total length (as an approximate value) based on the two-dimensional coordinates of the points constituting the contact trajectory (4) and an appropriate statistical calculation method. For example, the contact trajectory (4) can be approximated to a part of an arc from coordinates of the center of the base of the thumb (31), the starting point, and the ending point, and a length of the portion of the arc can be calculated as a total length of the contact trajectory (4). In addition, the length (411) may be the shortest distance between both end points of the contact trajectory (4), that is, the starting point and the ending point (length of a straight line connecting the starting point and the ending point). In this case, the application (2) calculates the shortest distance from the two-dimensional coordinates of the starting point and the ending point of the contact trajectory (4). In FIG. 4, La (total length) and Ls (distance between the starting point and the ending point) are shown as the length (411).

For example, a turning angle (412) of the thumb (31) can be measured from the contact trajectory (4) as a feature amount expressing the dimension (41). Since the thumb (31) slides on the touch panel (11) while turning around the base of the thumb (31), the contact trajectory (4) is usually approximated to a fan-shaped periphery. Therefore, an angle of an approximated fan shape can be calculated as the turning angle (412) from the coordinates of contact points belonging to the contact trajectory (4). FIG. 4 schematically shows an aspect of calculating the turning angle (412) as an angle θ for a contact trajectory (4).

When the subject (3) performs drum rolling of the scale on the touch panel (11) in Step 2 described above, in Step 3, a scale movement (413) before and after the drum rolling in Step 2 is measured as the dimension (41). In this case, instead of sensing the entire movement trajectory of the thumb (31) for detection as the contact trajectory (4), the scale movement (413) reflecting a sliding speed and a pressing force of the thumb (31) at a drum roll point is regarded as the contact trajectory (4). This is based on a hypothesis that when the sliding speed and the pressing force of the thumb (31) at the drum roll point are large, the contact trajectory (4) generated as a result of continuing the sliding motion is also lengthened.

When the mental and physical condition of the subject (3) is good or positive and active, the strength of the sliding motion of the thumb is increased, or the scale is vigorously drum-rolled on the ball (32) of the thumb (31), and any of the length (411), the turning angle (412), and the scale movement (413) measured as the dimension (41) has a large value.

### Step 4 of Test Method (100)

In Step 4, the application (2) compares the dimension (41) with a preset reference value (40) to generate answer data (5) of the subject (3) with respect to the question and/or invitation message (21). Usually, the answer data (5) is generated by the following rule.
When the dimension (41) is equal to or larger than the reference value (40), it is determined that the subject (3) has affirmed (answered “Yes”) the question and/or invitation message (21).
When the dimension (41) is smaller than the reference value (40), it is determined that the subject (3) has denied the question and/or invitation message (21) (answered “No”).

The application (2) can display an answer content on the touch panel (11) based on the generated answer data (5). For example, when the affirmative answer data (5) is generated, a text “Your answer is ”YES“.” can be displayed on the display screen (1101) of the smartphone (101). In addition, for example, when the negative answer data (5) is generated, a text “Your answer is “NO”.” can be displayed on the display screen (1101) of the smartphone (101).

### Step 5 of Test Method (100)

In Step 5, the application (2) determines a mental and physical condition (7) of the subject (3) based on performance data (6) calculated from one or more pieces of answer data (5) obtained by executing Steps 1 to 4 one or more times.

The application (2) can display the determination result on the touch panel (11).

For example, when the subject (3) responds to one question and/or invitation message (21), the positive answer data (5) is generated, and thus a message: “You look energetic today. Keep it up!” is displayed, and when the negative answer data (5) is generated, a message “You might be tired today. Would you like to take a rest?” can be displayed.

A method of calculating the performance data (6) from a plurality of pieces of the answer data (5) is not limited. For example, the sum of all pieces of the answer data (5) can be used as the performance data (6). Furthermore, for example, each answer data (5) may be weighted, and a weighted sum or a weighted average of the answer data (5) may be used as the performance data (6). The application (2) can determine “good” or “not good” of the mental and physical condition (7) by comparing the performance data (6) of the subject (3) with the preset reference value (61). In general, the determination is made by the following rule.
When the performance data (6) is equal to or larger than the reference value (61), it is determined that the mental and physical condition (7) is good.
When the performance data (6) is smaller than the reference value (61), it is determined that the mental and physical condition (7) is not good.

The application (2) can store each question and/or invitation message (21), each contact trajectory (4), and each answer data (5) on a database. The database may be a so-called local site on the portable terminal (1) or may be a site provided in a cloud on the Internet.

In the test method of the present invention, a test history of the subject (3) can be stored as a test history data set in association with information such as an age and a gender of the subject (3), a test date and time, and position and weather information of the portable terminal (1) at the time of the test. In the test method of the present invention, the test history can be updated whenever the test history is accumulated and whenever the information of the subject (3) is changed.

The application (2) can redisplay a determination result of a past contact trajectory (4) or a determination result of a past mental and physical condition (7) on the touch panel (11) in response to request information input to the portable terminal (1) by the subject (3). The application (2) can detect a change in the determination result of the mental and physical condition (7) of the subject (3) and display a message giving a notification of the change on the portable terminal (1). In this case, for example, a text message “Improvement of the mental and physical condition is observed. Keep it up!”, “It seems that you are tired in the past week.” can be displayed, or a reading-out audio of the text can be output.

A user can observe and self-evaluate a mental and physical condition in a game-like manner by the test method of the present invention. Before and after each of Steps 1 to 5, known functions provided to individual users by an interface for playing a game or viewing an image by using a portable terminal device, for example, a menu selection, a request for screen clearing or re-execution, an information input, a so-called “favorite saving”, and the like can be added without limitation.

### EXAMPLES

### Example 1

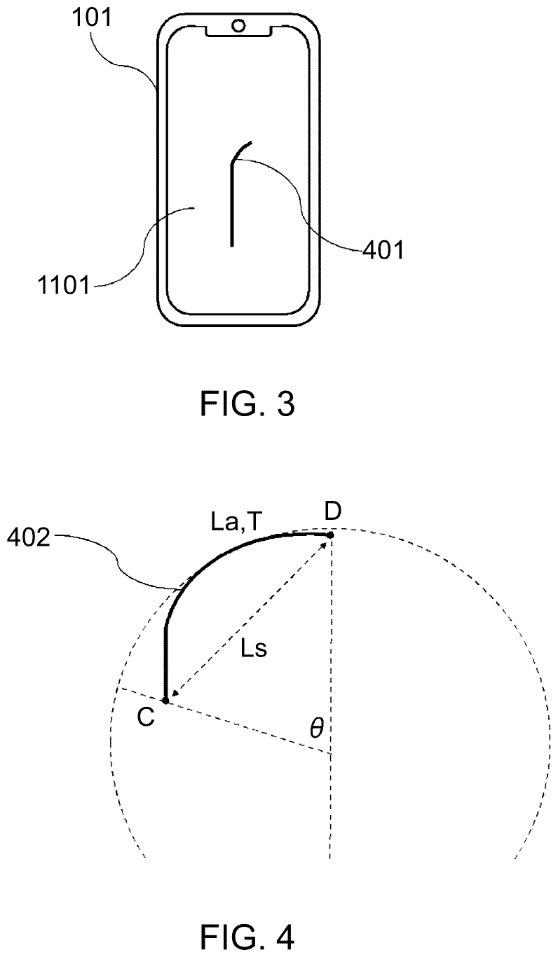

FIG. 3 schematically shows an example (401) of the contact trajectory (4) that is a measurement target in Step 3 of the present invention. FIG. 3 shows a case where the subject (3) is right-handed. There is an exaggeration or omission of details in FIG. 3.

The contact trajectory (401) is theoretically a line composed of a set of contact points between the ball (32) of the thumb (31) and the display screen (1101) which appear when the thumb (31) of the subject (3) is slid to flip up in a longitudinal direction of the display screen (1101) of the smartphone (101). In reality, there is a line drawing display output by the application (2) in response to a swipe of a finger on the display screen (1101).

### Example 2

FIG. 4 shows an example (402) of another contact trajectory (4). There is an exaggeration in FIG. 4. In FIG. 4, a starting point C and an ending point D of the contact trajectory (402) are emphasized. FIG. 4 shows a case where the subject (3) is right-handed. A dotted line is a straight line connecting the starting point C and the ending point D of the contact trajectory (402), and is an auxiliary line related to a fan shape that approximates the contact trajectory (402). The application (2) acquires the following numerical values with respect to the dimension (41) of the contact trajectory (402), and uses the numerical values as feature amounts for expressing a shape dimension (4102) of the contact trajectory (402).
La: A total length of the contact trajectory (402).
Ls: The shortest distance between the starting point C and the ending point D.
θ: A turning angle of the thumb (31)
T: Movement time of the thumb (31) (time from departure at point C to arrival at point D)

The application (2) determines the mental and physical condition of the subject (3) based on a result of comparing each of the feature amounts La, Ls, θ, and T with a preset threshold.

### Example 3

Example 3 is a case where the test is performed on the same invitation message (21) generated by the application (2) for the subject (3) at an interval of one week. FIG.
5 shows an aspect in which the application (2) displays past three times of determination results (73, 74, and 75) of the mental and physical condition on the smartphone (101). Four or more times of results may be displayed. The determination results (73, 74, and 75) are obtained by comparing the feature amounts acquired in association with each of the contact trajectories (403, 404, and 405) (not shown) at each time with a threshold, and marking a mental and physical level (good or bad) determined based on a certain rule. The more the number of face marks, the better the mental and physical condition.

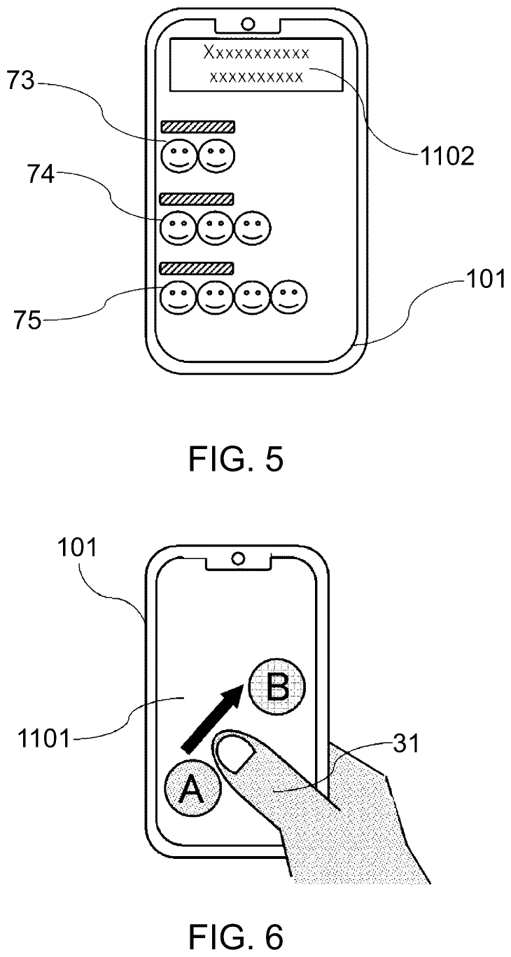

The determination result (73) is acquired on * (month) 1, **** (year). The determination result (74) is acquired on * (month) 8, **** (year). The determination result (75) is acquired on * (month) 15, **** (year). As shown in FIG. 5, during the three test periods, the mental and physical condition of the subject (3) is improved. In this case, the application (2) can display a text “You are gaining confidence. Let's keep it up” as a message (1102) to the smartphone (101).

When a new contact trajectory (406) (not shown) and a determination result (76) are acquired, the entire three times of contact trajectories (403, 404, 405) move upward (to approach the message (1102) in FIG. 5), and the latest determination result (76) is displayed below the determination result (75). A contact trajectory acquired next is also displayed in the same manner. The subject (3) can make a comparison of the shape dimension of the past three times of contact trajectory.

### Example 4

Example 4 is an example in which the drum roll method is used in Step 2 and Step 3. FIG. 8 shows an aspect in which the drum roll starts in Step 2 of a first test.
FIG. 8 includes an exaggeration or omission of details. In the state shown in FIG. 8, the thumb (31) of the subject (3) is in contact with a scale “0” which is the starting point.
The display screen (1101) also displays a scale “1” adjacent to the scale “0”. The thumb (31) starts sliding in an arrow direction (in a vertical direction when viewed from the subject (3)). The drum roll for the scale starts.

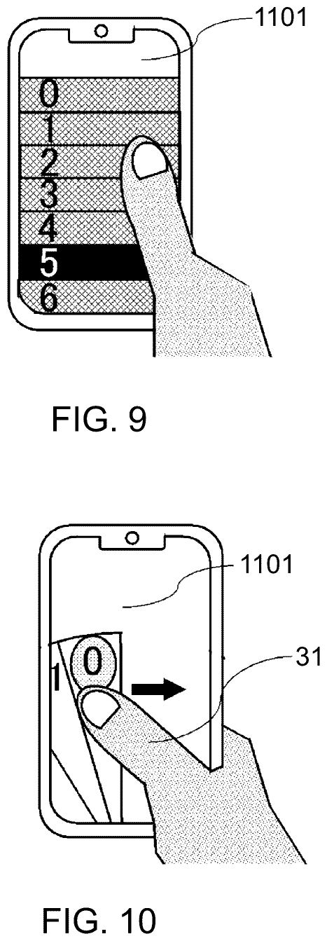

Thereafter, when the drum roll for the scale is stopped, the display screen (1101) is stabilized in a state shown in FIG. 9. For the subject (3), it seems that the scale “0” is pushed up with the thumb (31) such that the entire scales are rotated to a state in FIG. 9, and a scale “5” which is not visible at the time of the drum roll start is pulled up to a position of the scale “0”. In the first test, “5” is measured as a scale movement (413). In the test, the answer data (5) indicating “good” is generated by comparison with a preset reference scale movement (4150)3. The test is performed three times a day in the same manner to acquire the scale movement (413) and the answer data (5), and the change in the answer data (5) for one week can be displayed.

### Example 5

Example 5 is an example in which the drum roll display of Example 4 is modified into a fan shape. FIG. 10 shows an aspect in which the drum roll starts. In Example 5, the thumb (31) starts to slide in an arrow direction. The drum roll for the scale starts. When the thumb (31) starts to slide in the arrow direction (a horizontal direction when viewed from the subject (3)), the drum roll of the scale starts.

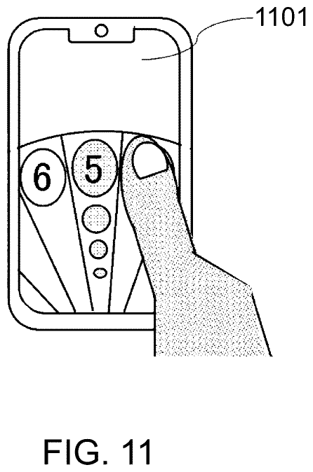

Thereafter, when the drum roll of the scale is stopped, the display screen (1101) is stabilized in a state shown in FIG. 11. “5” is measured as the scale movement (413).

### INDUSTRIAL APPLICABILITY

According to the present invention, a test that replaces the O-ring test can be easily performed by anyone, anytime, anywhere. The test method of the present invention does not require a person other than the subject. Therefore, the subject can take the test with ease and without stress, and enjoy the test. That is, the present invention can be said to be a method for performing the O-ring test with a game-like manner. Therefore, the present invention provides a service product that is attractive to the health promotion industry.

### REFERENCE SIGNS LIST

101 Smartphone
1101 Display screen of smartphone
31 Thumb
32 Ball of thumb
401 Contact trajectory
402 Contact trajectory
73, 74, 75 Determination result
1102 Text display

1. A test method using a portable terminal (1) and an application (2), the test method comprising:
Step 1 in which the application (2) starts an operation for a test;
Step 2 in which a subject (3) responds to a question and/or invitation message (21), and then slides a ball (32) of a thumb (31) of a dominant hand of the subject (3) on a surface of a touch panel (11), the touch panel (11) having a display function and a sensor function; and
Step 3 in which the application (2) measures a dimension (41) of a contact trajectory (4) between the ball (32) of the thumb (31) that appears in Step 2 and the touch panel (11).

2. The test method according to claim 1, wherein
in Step 1, the application (2) outputs the question and/or invitation message (21) for making a request for a response of the subject (3), and
the test method further comprises after Step 3:
Step 4 in which the application (2) compares the dimension (41) with a preset reference value (40) to generate answer data (5) of the subject (3) to the question and/or invitation message (21).

3. The test method according to claim 1, wherein
in Step 1, the application (2) outputs the question and/or invitation message (21) for making a request for a response of the subject (3), and
the test method further comprises after Step 3:
Step 4 in which the application (2) compares the dimension (41) with a preset reference value (40) to generate answer data (5) of the subject (3) to the question and/or invitation message (21); and
Step 5 in which the application (2) determines a mental and physical condition (7) of the subject (3) based on performance data (6) calculated from one or more pieces of answer data (5) obtained by executing Steps 1 to 4 one or more times.

4. The test method according to claim 1, wherein
in Step 3, a length (411) of the contact trajectory (4) is measured as the dimension (41), and
in Step 4, the application (2) compares the length (411) with a preset reference length (4110) to generate answer data (5) of the subject (3) to the question and/or invitation message (21).

5. The test method according to claim 4, wherein
the length (411) corresponds to a total length of the contact trajectory (4).

6. The test method according to claim 4, wherein
the length (411) corresponds to a shortest distance between both end points of the contact trajectory (4).

7. The test method according to claim 1, wherein
in Step 3, a turning angle (412) of the thumb (31) based on the contact trajectory (4) is measured as the dimension (41), and
in Step 4, the application (2) compares the turning angle (412) with a preset reference turning angle (4140) to generate answer data (5) of the subject (3) to the question and/or invitation message (21).

8. The test method according to claim 1, wherein
in Step 2, the subject (3) responds to the question and/or invitation message (21), and then slides the ball (32) of the thumb (31) of the dominant hand of the subject (3) on the surface of the touch panel (11) to drum roll a scale displayed on the touch panel (11),
in Step 3, a scale movement (413) by the drum roll in Step 2 is measured as the dimension (41), and
in Step 4, the application (2) compares the scale movement (413) with a preset reference scale movement (4150) to generate answer data (5) of the subject (3) to the question and/or invitation message (21).
